(19) United States
(12) Patent Application Publication
(10) Pub. No.: US 2025/0380718 A1
(43) Pub. Date: Dec. 18, 2025

(54) METHODS OF FORMING COMESTIBLE CELL-BASED FOOD PRODUCTS UTILIZING HIGH-MOISTURE EXTRUSION

(71) Applicant: Upside Foods, Inc., Berkeley, CA (US)

(72) Inventors: Ibrahim Marjieh, Oakland, CA (US); John Mampra Mathew, Plano, TX (US)

(21) Appl. No.: 18/744,309

(22) Filed: Jun. 14, 2024

Related U.S. Application Data
(63) Continuation of application No. 18/742,966, filed on Jun. 13, 2024.

(51) Int. Cl. A23J 3/26; A23J 3/14; A23J 3/22; A23L 13/40; A23L 13/75; C12N 5/07

(52) U.S. Cl. CPC A23J 3/26; A23J 3/14; A23J 3/227; A23L 13/426; A23L 13/428; A23L 13/75; C12N 5/06

(57) ABSTRACT

This disclosure describes methods of forming an extruded cell-based food product that mimics the texture and flavor of slaughtered meat. Generally, the disclosed method comprises cultivating non-human animal cells. The disclosed methods combine the non-human cultivated animal cells with one or more plant proteins. In some embodiments, the disclosed methods also add one or more amino acids and/or a marinade to the non-human cultivated animal cells and the one or more plant proteins. In some embodiments, the disclosed methods extrude the non-human cultivated animal cells and one or more plant proteins utilizing high-moisture extrusion to form an extruded cell-based food product with a fibrous texture.

### CROSS-REFERENCE TO RELATED APPLICATIONS

The present application is a continuation of U.S. application Ser. No. 18/742,966, filed on Jun. 13, 2024. The aforementioned application is hereby incorporated by reference in its entirety.

### BACKGROUND

As the world's population continues to grow, cell-based or cultured meat products for consumption have emerged as an attractive alternative (or supplement) to conventional meat from slaughtered animals. For instance, cell-based, cultivated, or cultured meat represents a technology that could address the specific dietary needs of humans. Cell-based meat products can be prepared from one or more of cultivated adherent or suspension cells derived from a non-human animal. The cells for cell-based meat are grown in a food cultivation facility and are formed and shaped to mimic familiar forms of conventional slaughtered meat. Cell-based meat may meet or even exceed conventional slaughtered meat in terms of nutrition, flavor, and health impact.

In addition to addressing dietary needs, cell-based-meat products help alleviate several drawbacks linked to conventional meat products for humans, livestock, and the environment. For instance, conventional slaughtered meat production involves controversial practices associated with animal husbandry, slaughter, and harvesting. Other drawbacks associated with slaughtered meat production include low conversion of caloric input to edible nutrients, microbial contamination of the product, emergence and propagation of veterinary and zoonotic diseases, relative natural resource requirements, and resultant industrial pollutants, such as greenhouse gas emissions and nitrogen waste streams.

Despite advances in creating cell-based-meat products, existing methods for cultivating and processing cell-based-meat products face several shortcomings, such as challenges or failures to mimic the textures, appearance, and flavors of slaughtered meat. Existing methods often produce cell-based-meat products with undesirable textures and flavors. For instance, certain systems grow cells in a pure single cell suspension. While the cells grow in the pure single cell suspension, they are typically unable to mimic multicellular structures. Thus, suspension grown cells are often too soft and lack the fibrous structure, i.e., the grain, found in conventionally slaughtered meat.

These, along with additional problems and issues are present in existing methods for cultivating cell-based-meat products.

### BRIEF SUMMARY

This disclosure generally describes methods and apparatuses for creating cell-based food products (e.g., cell-based meat products) through high-moisture extrusion with texture that mimics the texture of conventional slaughtered meat. For example, the disclosed methods include cultivating non-human animal cells in suspension and combining the non-human cultivated animal suspension cells with one or more plant proteins and an amino acid (e.g., glutamic acid). In some cases, the disclosed methods form a comestible cell-based food product by extruding the non-human cultivated animal cells, one or more plant proteins, and the amino acid. For example, in one or more embodiments, the disclosed methods form a comestible cell-based food product by combining the non-human cultivated animal cells, the one or more plant proteins, and glutamic acid while regulating the temperature, moisture content, viscosity, pressure, and shear forces within the extruder. In one or more embodiments, the disclosed methods extrude the non-human cultivated animal cells, one or more plant proteins and glutamic acid into a cooling die. By passing through the cooling die, the disclosed method can form the non-human cultivated animal cells, one or more plant proteins, and glutamic acid into a comestible cell-based food product with a texture similar to that of conventional slaughtered meat.

Additional features and advantages of one or more embodiments of the present disclosure will be set forth in the description that follows.

### BRIEF DESCRIPTION OF THE DRAWINGS

Various embodiments will be described and explained with additional specificity and detail through the use of the accompanying drawings, which are summarized below.

FIG. 1 illustrates an overview diagram of forming an extruded cell-based food product from non-human cultivated animal cells and one or more plant proteins through high-moisture extrusion in accordance with one or more embodiments of the present disclosure.

FIG. 2 illustrates a flow chart of adding non-human cultivated animal cells, one or more plant proteins, glutamic acid, and/or marinade to an extruder in accordance with one or more embodiments of the present disclosure.

FIG. 3 illustrates an overview diagram of combining non-human cultivated animal cells, one or more plant proteins, amino acid, and/or marinade while regulating a temperature of a barrel of an extruder in accordance with one or more embodiments of the present disclosure.

FIG. 4 illustrates an overview diagram of a temperature profile of an extruder during high-moisture extrusion in accordance with one or more embodiments of the present disclosure.

FIG. 5 illustrates an overview diagram of extruding a comestible cell-based food product with a plurality of fibers extending in a non-parallel direction relative to the direction of extrusion in accordance with one or more embodiments of the present disclosure.

FIGS. 6A-6B illustrate the shape and texture of an extruded cell-based food product in accordance with one or more embodiments of the present disclosure.

FIG. 7 illustrates an overview diagram of extruding a cell-based food product through a cooling die with a shape of chicken breast in accordance with one or more embodiments of the present disclosure.

FIG. 8 illustrates a chart comparing properties of the extruded comestible cell-based food product and conventional meat products in accordance with one or more embodiments of the present disclosure.

FIG. 9 illustrates series of acts for forming a comestible cell-based food product through high-moisture extrusion in accordance with one or more embodiments of the present disclosure.

FIGS. 10A-10B illustrate a sequence diagram of growing and processing different types of cells in accordance with one or more embodiments of the present disclosure.

### DETAILED DESCRIPTION

This disclosure describes one or more embodiments of creating a cultivated cell-based food product through high-moisture extrusion. Generally, the disclosed method comprises cultivating a plurality of non-human animal cells and adding the cultivated non-human animal cells along with one or more plant proteins to an extruder. The disclosed methods pass the non-human cultivated animal cells and one or more plant proteins through the extruder into a cooling die and create a plurality of fibers. In certain implementations, the disclosed method combine the cultivated non-human animal cells and one or more plant proteins with one or more amino or food acids in a manner that synergistically improves the texture of an extruded cell-based food product. In some embodiments, the disclosed methods include adding marinade to the cultivated non-human animal cells after the temperature of the barrel drops from a peak temperature. The disclosed methods can pass the marinated non-human cultivated animal cells and one or more plant proteins through a cooling die and form an extruded cell-based food product. Additionally, the disclosed methods can include extruding the non-human cultivated animal cells and one or more plant proteins in a manner that creates fibers extending in a non-parallel direction relative to a direction of extrusion.

As mentioned, the disclosed methods comprise cultivating a plurality of non-human animal cells in suspension. In some embodiments, the plurality of non-human animal cells is grown in a growth medium until they reach a specified density or for a specified period of time. In one or more cases, the non-human cultivated animal cells are grown in animal component free growth medium.

The disclosed methods further include combining the plurality of non-human cultivated animal cells, one or more plant proteins, and one or more amino or food acids. For example, the disclosed methods can add the non-human cultivated animal cells, one or more plant proteins, and glutamic acid to an extruder and form a cell-based dough by mixing the non-human cultivated animal cells, one or more plant proteins, and glutamic acid with one or more screws within a barrel of the extruder. In one or more embodiments, the disclosed methods can regulate various factors of the extruder (e.g., temperature, pressure, moisture content, shear force) so that the non-human cultivated animal cells, one or more plant proteins, and glutamic acid form an extruded cell-based food product with an improved texture.

As indicated above, the disclosed methods can form an extruded cell-based food product comprising extruded fibers of the non-human cultivated animal cells, one or more plant proteins, and glutamic acid and/or marinade. In particular, the disclosed methods can extrude the non-human cultivated animal cells, one or more plant proteins, glutamic acid and/or marinade through a cooling die in a manner that creates a plurality of fibers that extend in a non-parallel direction relative to a direction of extrusion.

As indicated above, the disclosed methods provide several benefits relative to existing methods for forming cultivated cell-based-meat products through high-moisture extrusion. In particular, the disclosed methods form an extruded cell-based food product with improved texture and flavor to existing methods. For example, the combination of the non-human cultivated animal cells, one or more plant proteins, and glutamic acid can synergistically improve the texture of the extruded cell-based food product. In particular, prior to adding glutamic acid to the non-human cultivated animal cells and one or more plant proteins, the extruded fibers have a rubbery texture. Addition of glutamic acid to the one or more plant proteins and the non-human cultivated animal cells resulted in a dramatic improvement to the texture of the extruded cell-based food product. In particular, adding glutamic acid to the non-human cultivated animal cells and the one or more plant proteins resulted in a fibrous, meat like texture, in terms of chewiness, and mouthfeel, achieving vastly more robust texture of the extruded cell-based food product. Alternative acids, such as other amino acids, lactic acid, citric acid, or any combination of these, also improve the texture of the extruded product to a similar extent. For instance, at a slightly lower pH, relative to ideal growing conditions for non-human cultivated animal cells, the proteins in the non-human cultivated animal cells and/or plant proteins have a more neutral charge, which increases the ability of the desaturated protein changes to align and create a more organized and improved texture. By forming and extruded cell-based food product with non-human cultivated animal cells, one or more plant proteins, and acid, the disclosed methods create a material that more closely mimics the texture characteristics of a target slaughtered meat.

Furthermore, in some cases, the disclosed methods can improve the flavor of cell-based-food products. For example, adding the glutamic acid to the non-human cultivated animal cells and the one or more plant proteins improved the umami flavor of the extruded cell-based food product. As another example, lactic acid provides less umami flavor, achieving the desired umami level extruded cell-based food product by combining glutamic acid with lactic acid. In another example, glutamic acid can be replaced with more lactic acid to target a more beef like flavor. Relatedly, the disclosed method can further improve the flavor of cell-based products by adding marinade to the non-human cultivated animal cells and one or more plant proteins after reaching a peak temperature of a barrel within the extruder. Some existing methods coat an extruded food product with marinade after extrusion to cover undesirable beany off-flavors in the final product. However, such methods are unable to disperse marinade throughout the product, e.g. unable to reach the center, resulting in the beany off-flavor remaining in the middle of the extruded food product. Unlike such methods, the disclosed method can mix the marinade within the barrel of the extruder after reaching a peak temperature and create a more homogenized distribution of the marinade throughout the extruded cell-based food product.

Additionally, the extruded cell-based food product can imitate the appearance of muscle fibers found in slaughtered target meat. For example, the disclosed methods can form a plurality of fibers in the extruded cell-based food product and arrange the fibers in a manner that resembles the grain and/or structure of conventional cuts of meat (e.g., chicken breast, sliced ham, etc.).

As illustrated by the foregoing discussion, the present disclosure utilizes a variety of terms to describe features and advantages of the disclosed methods. Additional detail is now provided regarding the meaning of such terms. As used herein, the term “cells” (or “non-human cultivated animal cells”) refers to cells that form meat. Generally, non-human cultivated animal cells may comprise at least one of muscle cells, muscle progenitor cells, or muscle support cells. In particular, non-human cultivated animal cells may comprise different cell types, such as one or more of myoblasts, mesangioblasts, myofibroblasts, mesenchymal stem cells, embryonic stem cells, hepatocytes, fibroblasts, pericytes, adipocytes, epithelial, chondrocytes, osteoblasts, osteoclasts, pluripotent cells, somatic stem cells, endothelial cells, or other similar cell types. Furthermore, cells may comprise different types of progenitor cells, including myogenic progeny and progenitors, adipogenic progeny or progenitors, mesenchymal progeny or progenitors, or other types of progenitor cells. In some instances, the cells may comprise cells from distinct lineages, such as ectoderm or endoderm lineages, that have been transdifferentiated into cells useful for forming a cell-based food product for consumption, such as those cell types described above.

As used herein, the term “suspension culture” (or “suspension”) refers to cells growing in an at least partially liquid growth medium in which cells grow, multiply, and/or maintain nourishment. In particular, a suspension includes an agitated growth medium that is housed in a container in which single cells or small aggregates of cells grow, multiply, and/or maintain nourishment from the nutrients of the agitated growth medium. Cells grown in suspension are not attached to a substrate and therefore differ from an adherent culture.

Also, as used herein, the terms “cell culture media” or “culture media” refer to a liquid or gel comprising compounds that support the growth of cells. In particular, cell culture media comprises sources of energy and compounds to regulate the cell cycle. For example, a cell culture media can contain amino acids, vitamins, inorganic salts, glucose, dissolved gases, serum, growth factors, hormones, and attachment factors. The cell media may also help maintain pH and osmolarity during cell growth and proliferation.

As used herein, the term “plant protein” refers to a protein derived from a plant. For example, a plant protein can include complete proteins (comprising all nine essential amino acids) or incomplete proteins (comprising fewer than the nine essential amino acids). In some embodiments, the plant protein can be a protein isolate, a protein concentrate, or some combination thereof. Example plant proteins can come from nuts, beans, legumes, soybeans, quinoa, wheat, rice, seeds, lentils, or peas.

As used herein, the term “extruder” refers to a machine that applies shear forces, pressure changes, and temperature changes to a mixture as it travels through a barrel. The mixture is conveyed by a force of one or more screws spinning and pushing the material through different temperature zones, while maintaining pressure and shear forces according to screw configuration. In one or more embodiments, the screws manipulate the mixture. In some cases, the screws can have various screw elements to promote mixing, kneading, and/or twisting the non-human cultivated animal cells, one or more plant proteins, glutamic acid, and/or marinade while in the barrel of the extruder. Relatedly, different screw elements can correspond with temperature, pressure, moisture content, and shear forces exerted on the non-human cultivated animal cells and one or more plant proteins. In certain implementations, the extruder can include various ports for adding ingredients, such as the non-human cultivated animal cells, one or more plant proteins, glutamic acid, oil, and/or marinade. In some cases, the extruder can include a motor dictating the speed of the one or more screws.

As used herein, the term “acid” refers to a chemical substance that can donate a proton (H⁺ ion) or form a covalent bond with an electron pair in a reaction. Acids are characterized by their ability to increase the concentration of hydrogen ions (H⁺) in an aqueous solution, enabling pH values less than 7. Common properties of acids include a sour taste, the ability to turn blue litmus paper red, and reactivity with bases to form salts and water. Acids play a critical role in various chemical reactions and industrial processes.

As used herein, the term “food acid” refers to acids that occur naturally in foods or are added to them to impart a sour or tart taste, act as preservatives, or maintain/modify the pH balance. Food acids are generally safe for consumption and play important roles in food processing and flavoring. Example food acids include

As used herein, the term “amino acid” refers to organic compounds that constitute the fundamental building blocks of proteins. An amino acid molecule comprises an amino group (—NH₂) and a carboxyl group (—COOH), along with a distinct side chain (R citric acid, acetic acid, lactic acid, tartaric acid, malic acid, phosphoric acid, ascorbic acid, fumaric acid, sorbic acid, and benzoic acid. group) that imparts unique properties and functions to the amino acid. Amino acids, commonly incorporated into proteins, play a crucial role in various biological processes, including protein synthesis, enzymatic activity, and metabolic pathways. Amino acids are categorized into essential amino acids, which must be acquired through dietary intake, and non-essential amino acids, which can be synthesized endogenously by the organism. Example amino acids include alanine (Ala), arginine (Arg), asparagine (Asn), aspartic acid (Asp), cysteine (Cys), glutamic acid (Glu), glutamine (Gln), glycine (Gly), histidine (His), isoleucine (Ile), leucine (Leu), lysine (Lys), methionine (Met), phenylalanine (Phe), proline (Pro), serine (Ser), threonine (Thr), and tryptophan (Trp).

As further used herein, the term “glutamic acid” refers to an amino acid. For example, glutamic acid can be the hydrochloride salt of glutamic acid (C₅H₉NO₄·HCL). In some cases, glutamic acid improves the umami flavor and texture of the extruded cell-based food product.

Relatedly, as used herein, the term “marinade” refers to flavor enriching mixture of spices, salts, flavorings, and/or liquids. In certain implementations, the marinade is a liquid that is added to the extruder prior to passing the non-human cultivated animal cells and one or more plant proteins through the cooling die. In one or more embodiments, the marinade can be distributed substantially throughout an extruded cell-based food product. Relatedly, the term “distributed substantially” refers to dispersing the marinade through the extruded cell-based food product without the presence of concentrated pockets of marinade in the extruded cell-based food product.

Relatedly, as used herein, the term “cooling die” refers to a die or mold through which the extruded non-human cultivated animal cells, one or more plant proteins, glutamic acid, and/or marinade can pass. In some cases, the cooling die is a mold with various heat zones for cooling and openings for regulating the temperature and/or pressure of the non-human cultivated animal cells, one or more plant proteins, glutamic acid, and/or marinade. In some embodiments, the cooling die has a single opening for which the non-human cultivated animal cells, one or more plant proteins, glutamic acid, and/or marinade can exit. In some embodiments, the cooling die can take on various shapes and/or dimensions.

As used herein, the term “target slaughtered meat” refers to a slaughtered meat with a structure targeted for imitation by a cell-based-meat product. In particular, a target slaughtered meat comprises muscle fiber bundles, typically organized into grains, and/or other muscle fiber structures organized in a particular way specific to the given slaughtered meat. For example, a target slaughtered meat may include red meat, poultry, or seafood. In some implementations, target slaughtered meat comprises processed slaughtered meat products like ham.

As used herein, the term “cell-based food product” refers to a food product comprising non-human animal cells grown in vitro. For instance, the cell-based food product can include isolated cells from animals combined with other ingredients or additives such as, but not limited to, plant proteins, salts, flavorings, acids. Such products are interchangeably referred to as in vitro meat product, in vitro food product, lab grown meat, cultured meat, cultured food, or slaughter free meat depending on context.

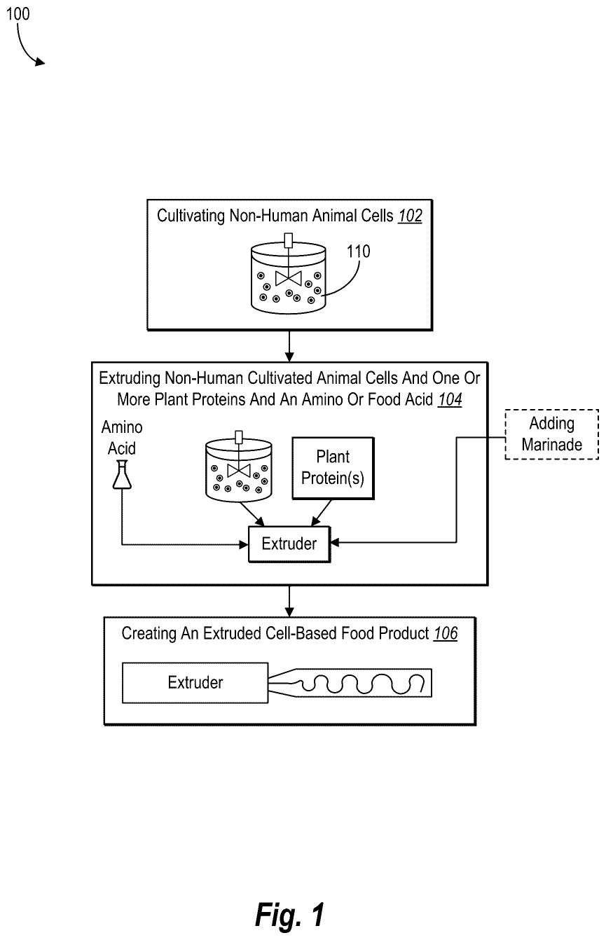

Additional detail will now be provided regarding the disclosed methods in relation to illustrative figures portraying example embodiments and implementations of the disclosed methods. FIG. 1 illustrates an overview of extruding a plurality of non-human cultivated animal cells, one or more plant proteins, amino or food acid and/or marinade in accordance with one or more embodiments of the present disclosure. By way of overview, FIG. 1 illustrates a series of acts 100 comprising an act 102 of cultivating non-human animal cells, an act 104 of extruding non-human cultivated animal cells and one or more plant proteins, and an amino or food acid, and an act 106 of creating a comestible cell-based food product utilizing an extruder.

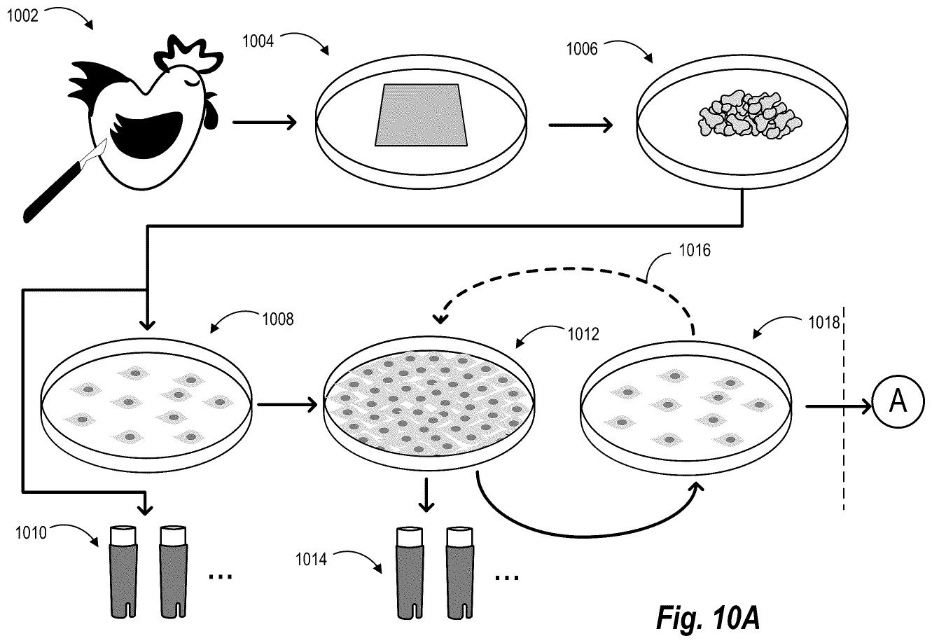

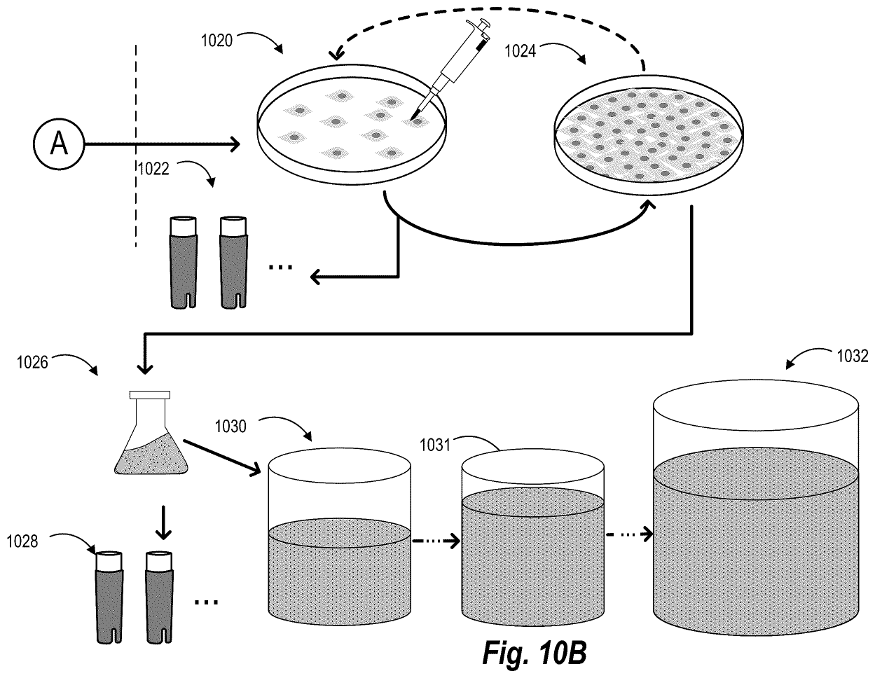

FIG. 1 illustrates the act 102 of cultivating non-human animal cells. As illustrated, the act 102 comprises growing the non-human cultivated animal cells 110 in suspension. In some cases, the non-human cultivated animal cells 110 are grown in cell culture media that supports cell growth, cell differentiation, or both. In some embodiments, the non-human cultivated animal cells can comprise cells of different cell types including myocytes, adipocytes, or fibroblasts. In some embodiments, the non-human cultivated animal cells may comprise cells of one or more cell types. FIGS. 10A-10B and the corresponding paragraphs detail cultivating cells in accordance with one or more embodiments.

As further shown in FIG. 1, the series of acts 100 further includes the act 104 of extruding non-human cultivated animal cells, one or more plant proteins, and one or more of an amino or food acid. More specifically, the act 104 comprises mixing a plurality of the non-human cultivated animal cells, one or more plant proteins, and an amino or food acid in an extruder. In some embodiments, the disclosed methods can combine the plurality of non-human cultivated animal cells, one or more plant proteins, and an amino or food acid according to a mixing ratio that results in an extruded cell-based food product having a texture, composition, and/or nutritional profile similar to the solid content composition and/or nutritional profile of a target slaughtered meat (e.g., chicken, beef, pork, fish, etc.). As also shown in FIG. 1, the disclosed methods can combine marinade to the non-human cultivated animal cells and one or more plant proteins within the extruder.

As further illustrated in FIG. 1, the disclosed methods include the act 106 of forming an extruded cell-based food product. The act 106 comprises extruding the plurality of non-human cultivated animal cells, the one or more plant proteins, the amino acid, and/or the marinade through a cooling die. In some embodiments, the disclosed methods can extrude the non-human cultivated animal cells, one or more plant proteins, the amino acid and/or marinade at a predetermined extrusion rate. In some embodiments, the disclosed methods can extrude the non-human cultivated animal cells and the one or more plant proteins in a manner that creates a plurality of fibers. Relatedly, the plurality of fibers can leave the extruder and enter the cooling die in a non-parallel direction relative to a direction of extrusion. In one or more implementations, the disclosed methods can form the extruded cell-based food product into a cutlet with a desired shape.

In one or more implementations, the extruded cell-based food product, unless otherwise manipulated to include, does not include vascular tissues, such as veins and arteries, whereas conventional meat does contain such vasculature, and contains the blood found in the vasculature. Accordingly, in some implementations, the comestible cell-based food product does not comprise any vasculature. Likewise, comestible cell-based food product, although composed of muscle or muscle-like tissues, unless otherwise manipulated to include, does not comprise functioning muscle tissue. Accordingly, in some implementations, the cell-based meat does not comprise functioning muscle tissue. It is noted that features such as vasculature and functional muscle tissue can be further engineered into the cell-based meat, should there be a desire to do so.

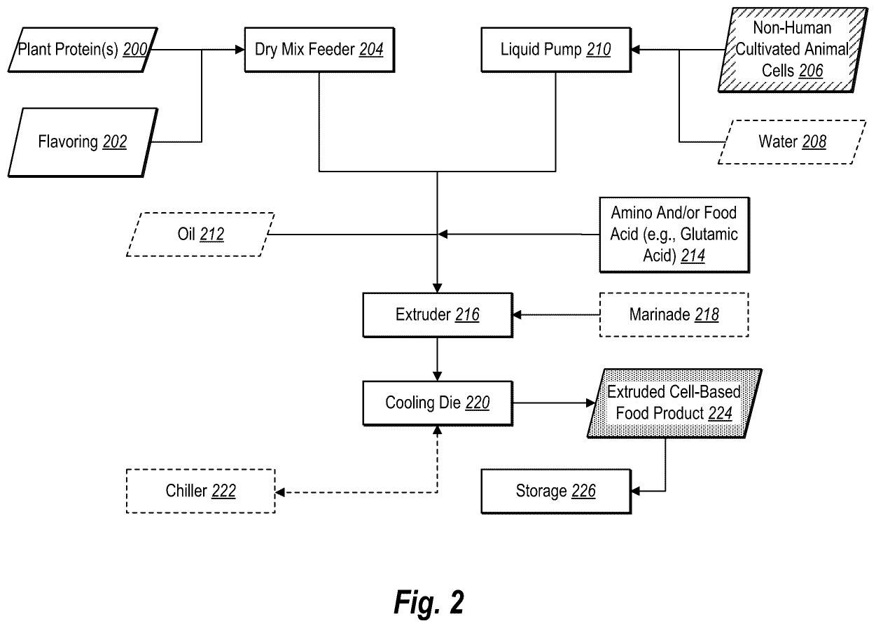

As described previously, the disclosed methods may combine a plurality of non-human cultivated animal cells, one or more plant proteins, and glutamic acid. In accordance with one or more embodiments, FIG. 2 illustrates an example method flow of combining the non-human cultivated animal cells and one or more plant proteins, and additional ingredients (e.g., glutamic acid, marinade, oil, flavor, and/or water) in an extruder in accordance with one or more embodiments.

As shown in FIG. 2, the disclosed methods can add one or more plant proteins (or more simply plant protein(s)) 200 and/or flavoring 202 to the extruder 216. In certain implementations, the plant protein(s) 200 can be isolates, concentrates, or some combination thereof. For instance, in certain embodiments, the plant protein(s) 200 can include soy protein isolate and/or wheat protein. In some cases, wheat protein can include wheat gluten. In one or more embodiments, the disclosed methods can add the plant protein(s) 200 based on percent weight of the extruded cell-based food product. For example, the plant protein(s) 200 can comprise between 25% and 50%, between 30% and 40%, and 30%, 40%, 40.8%, 45% or 50% of the weight of the extruded cell-based food product 224. For example, in one or more embodiments, the wheat protein can be between 25% and 40% by weight of the extruded cell-based food product 224. To further illustrate, in some implementations, the soy protein can be between 30% and 40% by weight of the extruded cell-based food product 224. As indicated above, the disclosed methods can combine the plant protein(s) 200 prior to adding the plant protein(s) 200 to the extruder 216.

As further shown in FIG. 2, the disclosed methods can add flavoring 202 to the extruder 216. In certain embodiments, the flavoring 202 can include various spices, salts, flavoring agents, and/or dry herbs. In one or more embodiments, the flavoring 202 can be between 0% and 5% weight of the extruded cell-based food product 224. As shown in FIG. 2, in one or more embodiments, the disclosed methods can add the flavoring 202 and plant protein(s) 200 to the extruder 216 through a dry mix feeder 204. In one or more embodiments, the dry mix feeder 204 is a feeding hopper that delivers the plant protein(s) 200 to the extruder 216.

As shown in FIG. 2, the disclosed method can add non-human cultivated animal cells 206 to the extruder 216 through a liquid pump 210. In one or more embodiments, the liquid pump 210 can inject the non-human cultivated animal cells 206 at a specific injection rate so that they create a homogenous composition (e.g., cell-based dough) when combined with the plant protein(s) 200.

As discussed above, in one or more cases, the disclosed method can combine the non-human cultivated animal cells 206 and the plant protein(s) 200 according to percent weight of the extruded cell-based food product 224. For example, in one or more embodiments, the percent weight of the non-human cultivated animal cells 206 in the extruded cell-based food product 224 can be between 1% and 60%, 10% and 50%, 20% and 40%, and 30%, 35%, 36.8%, 37%, or 40%.

As further shown in FIG. 2, in one or more cases, the disclosed method can add water 208 to the extruder 216 through the liquid pump 210. In certain embodiments, the percent weight of the water 208 added can be between 1% and 70%, 10% and 60%, 20% and 50%, 20% and 40%, and 15%, 18%, 20%, 30%, 50%, or 60%. In some embodiments, the percent weigh of the water 208 can match the percent weight of a target slaughtered meat. For example, the percent weight of water in conventional chicken is 70% and the percent weight of water of the extruded cell-based food product 224 can be 70%. Relatedly, in one or more embodiments, the disclosed methods can add the amount of water 208 based on the amount of non-human cultivated animal cells 206 or the moisture content of the extruded cell-based food product 224. For example, based on adding by weight 35% non-human cultivated animal cells to the plant protein(s) 200, the disclosed methods can add by weight 15% water 208 to the extruder 216. In some implementations, the water 208 can include one or more salts.

As further shown in FIG. 2, the disclosed methods can add oil 212 to the extruder 216. In certain cases, the oil 212 can be any plant-based oil, e.g. canola oil, coconut oil, corn oil, olive oil, palm oil, peanut oil, safflower oil, soybean oil, sunflower oil, vegetable oil, avocado oil, grapeseed oil, or any combination thereof. The disclosed method can add the oil 212, according to percent weight, based on the percent weight of the non-human cultivated animal cells 206 and/or the plant protein(s) 200. In one or more embodiments, the weight of the oil 212 can range between 0% to 10%, 0% to 5%, or 1%, 3%, or 4%.

As further shown in FIG. 2, in one or more embodiments, the disclosed methods can add one or more of a food or amino acid (e.g., glutamic acid 214) to the extruder 216. As described above, adding glutamic acid 214 to the non-human cultivated animal cells 206 and the plant protein(s) 200, improved the texture of the extruded cell-based food product 224. In particular, adding the glutamic acid hydrochloride improved the texture, whereas adding the sodium salt of glutamic acid (e.g. MSG) did not improve the texture of the extruded cell-based food product 224. Surprisingly a small amount of glutamic acid 214 changed the texture of the extruded cell-based food product 224 from soft and rubbery to hearty and more closely mimicking the texture of chicken. Indeed, in some embodiments, the glutamic acid 214 in combination with the non-human cultivated animal cells 206 and the plant protein(s) 200 synergistically improved the texture of the extruded cell-based food product 224 by reducing the rubberiness of the extruded cell-based food product 224. In certain cases, the glutamic acid 214 in combination with the non-human cultivated animal cells 206 and the plant protein(s) 200 can synergistically improve the texture of the extruded cell-based food product 224 by reducing the size of the fibers in the extruded cell-based food product 224. In some embodiments, the disclosed method can add glutamic acid 214 by a weight of 0.1% to 2%, 0.5% to 1%, or 0.2%, 0.35%, 1% of the extruded cell-based food product 224 to the extruder 216. Relatedly, the glutamic acid 214 can lower the pH of the non-human cultivated animal cells 206 and/or the plant protein(s) 200. For example, in some embodiments, the disclosed methods can lower the pH of the non-human cultivated animal cells 206, the plant protein(s) 200, and glutamic acid 214 to be between about 5.0 and 6.5. Relatedly, in certain cases, the disclosed method can add an amount of glutamic acid 214 that is sufficient to lower the pH of the extruded cell-based food product 224 to below 6.5. In some embodiments, the disclosed methods can add one or more amino acids and/or food acids to the extruder 216. In some embodiments, the amino acid can include glutamic acid 214, aspartic acid, leucine, etc. In certain cases, the disclosed method can add one or more amino acids and/or food acids to the extruder 216 to lower the pH of the extruded cell-based food product 224 to below 6.5.

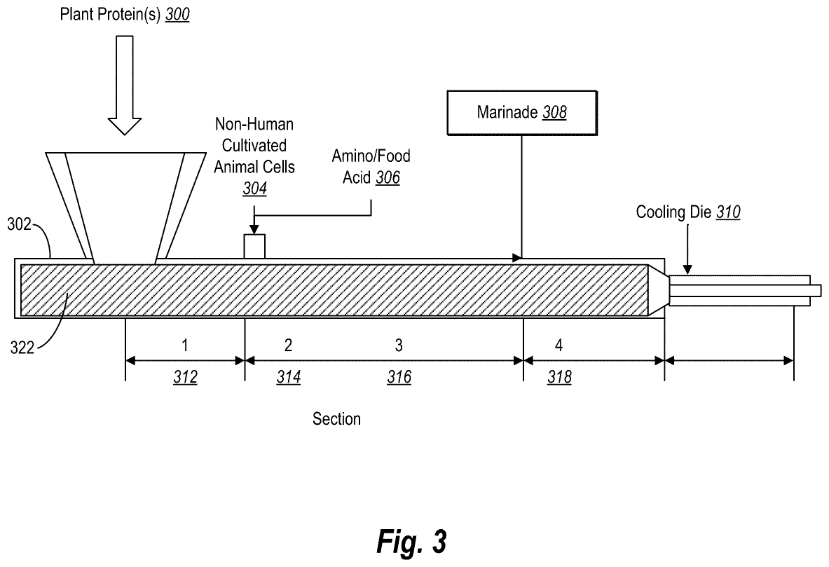

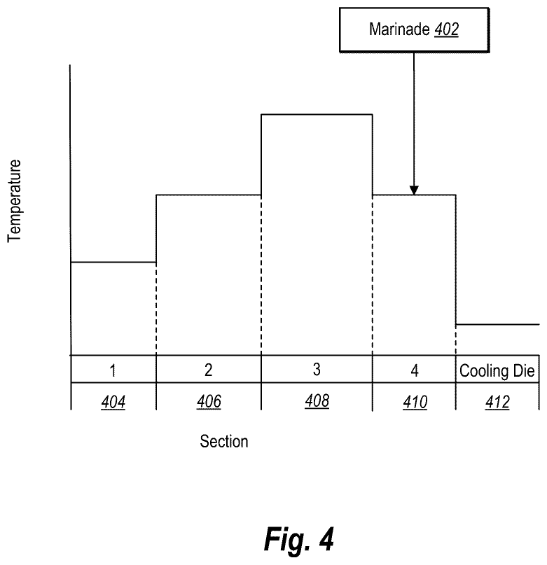

As indicated in FIG. 2, the disclosed methods can add a marinade 218 to the extruder 216. In some embodiments, the marinade 218 comprises 1% to 5% NaCl, 1% to 5% other flavorings, and 95% to 99% water. In some cases, the marinade 218 can be between 0.5% and 5% by weight of the extruded cell-based food product 224. In one or more embodiments, the disclosed methods can inject a liquid marinade 218 into the extruder 216 after the barrel of the extruder 216 reaches a peak temperature and starts decreasing. As discussed in more detail below with regard to FIG. 3 and FIG. 4, the disclosed method can regulate the temperature of the barrel of the extruder 216 and add the marinade 218 based on the temperature of the barrel so that the marinade 218 mixes into the combination of non-human cultivated animal cells 206 and/or the plant protein(s) 200 prior to passing the non-human cultivated animal cells 206 and/or the plant protein(s) 200 through a cooling die 220.

FIG. 2 further illustrates the disclosed method combining the non-human cultivated animal cells 206, plant protein(s), oil 212, glutamic acid 214, and/or marinade 218 in the extruder 216. As described in more detail below, the disclosed methods can adjust the temperature of the barrel, pressure within the barrel, the extrusion rate, moisture content, and/or screw speed of the extruder 216 to generate the extruded cell-based food product 224.

As further shown in FIG. 2, the disclosed methods can extrude the non-human cultivated animal cells 206, plant protein(s) 200, oil 212, glutamic acid 214, and/or marinade 218 into a cooling die 220. In some cases, the disclosed methods can extrude the non-human cultivated animal cells 206 and plant protein(s) 200 in a manner that creates a plurality of fibers where the fibers extend into the cooling die 220 in a non-parallel direction relative to a direction of extrusion. For example, the plurality of fibers can have a configuration within the cooling die 220 that is generally perpendicular to the direction of extrusion.

In certain implementations, the disclosed methods can control the temperature of the cooling die 220 with a chiller 222 to aid in the arrangement and tempering of the non-human cultivated animal cells 206 and/or the plant protein(s) 200. For example, the cooling die 220 can comprise various zones with one or more temperatures that can aid in the crosslinking of proteins within the extruded cell-based food product 224. In some embodiments, the chiller 222 can regulate the temperature of the cooling die 220 by circulating water around the cooling die 220.

As further shown in FIG. 2, the disclosed methods can form an extruded cell-based food product 224. In certain embodiments, the extruded cell-based food product 224 can include the non-human cultivated animal cells 206, plant protein(s), and glutamic acid 214. As described above, the glutamic acid 214 and plant protein(s) 200 synergistically improve the texture and/or flavor of the extruded cell-based food product 224. For example, the combination of the glutamic acid 214 and plant protein(s) 200 makes the texture of the extruded cell-based food product 224 more firm and akin to that of a target slaughtered meat (e.g., chicken, steak, ham). In some cases, the extruded cell-based food product 224 can include fibers that take on a non-parallel configuration relative to a direction of extrusion.

Finally, as shown in FIG. 2, the disclosed method can place the extruded cell-based food product 224 in storage 226. For example, the disclosed methods can place the extruded cell-based food product 224 in packaging and store the extruded cell-based food product 224 under refrigeration or freezing.

As discussed above, the disclosed methods may regulate the temperature of the barrel, pressure within the barrel, the extrusion rate, moisture content, and/or screw speed of the extruder to generate the extruded cell-based food product. FIG. 3 illustrates an example of combining non-human cultivated animal cells, one or more plant proteins, amino acid, and/or marinade while regulating a temperature, pressure, screw speed, and/or the extrusion rate of a barrel of an extruder and/or the moisture content of the one or more plant proteins and non-human cultivated animal cells in accordance with one or more embodiments of the present disclosure.

As shown in FIG. 3 and as indicated above, the disclosed methods can add plant protein(s) 300, non-human cultivated animal cells 304, an amino/food acid 306 (e.g., glutamic acid), and/or marinade 308 to an extruder 302. FIG. 3 illustrates a barrel 322 within the extruder 302. As shown in FIG. 3, the barrel 322 of the extruder 302 can include various sections for processing the plant protein(s) 300, non-human cultivated animal cells 304, an amino/food acid 306 and/or marinade 308. In one or more embodiments, the extruder 302 can regulate conditions of each section of the barrel 322 to process the plant protein(s) 300, non-human cultivated animal cells 304, amino/food acid 306, and/or marinade 308. For example, the disclosed methods can regulate the temperature, pressure, shearing forces, and/or extrusion rate of each section of the barrel 322 of the extruder 302. For example, in one or more embodiments, the screw speed of the barrel can range between 200-1000 rpm at 5-15 Kg/hr.

More specifically, in one or more embodiments each section of the extruder comprises a screw assembly that conveys, mixes, shears, kneads, reverse shears, reverse kneads, or reverse mixes the cell-based dough as it passes through the section of the extruder. Each extruder can comprise a single-screw configuration where each section comprises a single screw. Alternatively, the extruder comprises a twin-screw extruder where each section comprises a pair of screws. The cell-based dough is conveyed by mechanical pressure through the passage between rotating screw(s) and the stationary barrel by rotating the screw(s). As the cell-based dough is conveyed along the barrel, one or more ports can be used for injection of liquid ingredients, including cultivated animal cells, oil, water, marinade, or other ingredients. As the cell-based dough is conveyed to the end of the barrel, the cell-based dough exits the extruded and passes into a cooling die.

To reiterate, the sections of the extruder 302 can process the plant protein(s) 300, non-human cultivated animal cells 304, amino/food acid 306, oil, water, flavoring, and/or marinade 308. In one or more embodiments, each section of the extruder 302 correlates to a processing step of the plant protein(s) 300, non-human cultivated animal cells 304, an amino/food acid 306 (e.g., glutamic acid), and/or marinade 308. For example, a first section 312 can be an unheated feeding zone for mixing the plant protein(s) 300, non-human cultivated animal cells 304, amino/food acid 306, water, oil, and/or flavoring. As indicated below, the disclosed methods can mix non-human cultivated animal cells 304 and plant protein(s) 300 into a cell-based dough as they pass through the extruder 302.

As further shown in FIG. 3, in one or more cases, the disclosed methods can transport, with the screw and barrel 322, the cell-based dough into a second section 314. In certain embodiments, the second section 314 can increase the temperature and/or pressure of the barrel 322 and form the cell-based dough of the non-human cultivated animal cells 304 and plant protein(s) 300. For instance, in some cases, the pressure (e.g., die pressure) in the barrel can range between 5 psi and 200 psi, between 5 psi and 150 psi, between 10 psi and 100 psi, between 20 psi and 80 psi, or between 30 psi and 70 psi. In some instances, higher pressures can lead to better textures and higher pressures can be achieved by lowering flow rate, increasing an inner diameter of a cooling die, or both. In some embodiments, the cell-based dough can include the non-human cultivated animal cells 304, the plant protein(s) 300, amino/food acid 306, water, oil, and/or flavoring. As further shown in FIG. 3, the barrel of the extruder 302 can have a second section 314 with an increased temperature range and increased pressure. In one or more embodiments, the increased temperature, and/or pressure can unfold the proteins from the plant protein(s) 300 and/or the non-human cultivated animal cells 304 of the cell-based dough in the second section 314.

As further shown in FIG. 3, the extruder 302 can include a third section 316. In some embodiments, the third section 316 can further melt and/or denature the proteins from the plant protein(s) 300 and the non-human cultivated animal cells 304 of the cell-based dough by further increasing the temperature, shear forces, and/or pressure within the barrel 322. In some embodiments, the disclosed method reaches a peak temperature in the third section 316.

As FIG. 3 further illustrates, the disclosed methods can transport the cell-based dough into a fourth section 318 of the barrel 322 of the extruder 302. In one or more embodiments, the disclosed methods can lower the temperature of the barrel 322 in the fourth section 318. For instance, in some cases, when the barrel 322 of the extruder 302 reaches a peak temperature in the third section 316, the disclosed methods can decrease the temperature of the fourth section 318.

As further shown in FIG. 3, the disclosed methods can add marinade 308 to the cell-based dough after the barrel 322 of the extruder 302 drops from the peak temperature. In this manner, the disclosed methods can ensure that the marinade is not overheated/overcooked. As shown, since the cell-based dough has passed through the third section 316 of the extruder before the marinade is added, the cell-based dough is heated or cooked prior to addition of the marinade.

In one or more cases, adding the marinade 308 in the fourth section 318 can, with substantial uniformity, distribute the marinade 308 into the cell-based dough prior to passing through the cooling die 310. For example, as indicated above, adding the marinade 308 before the cell-based dough passes through the cooling die 310, prevents the marinade 308 from forming into concentrated pockets in the extruded cell-based food product. Relatedly, in some cases, the marinade 308 can have a greater concentration in the center of the extruded cell-based food product than the surface. For example, in some cases, based on the timing and/or location of injecting the marinade 308 in the fourth section 318, the marinade 308 can have a greater concentration in the center of the extruded cell-based product. In one or more embodiments, the marinade 308 can be between 1% and 5% by weight of the extruded cell-based food product. For example, based on the desired flavor and/or composition of the non-human cultivated animal cells 304 and plant protein(s) 300 of the extruded cell-based food product, the marinade 308 can be 4% by weight of the extruded cell-based food product.

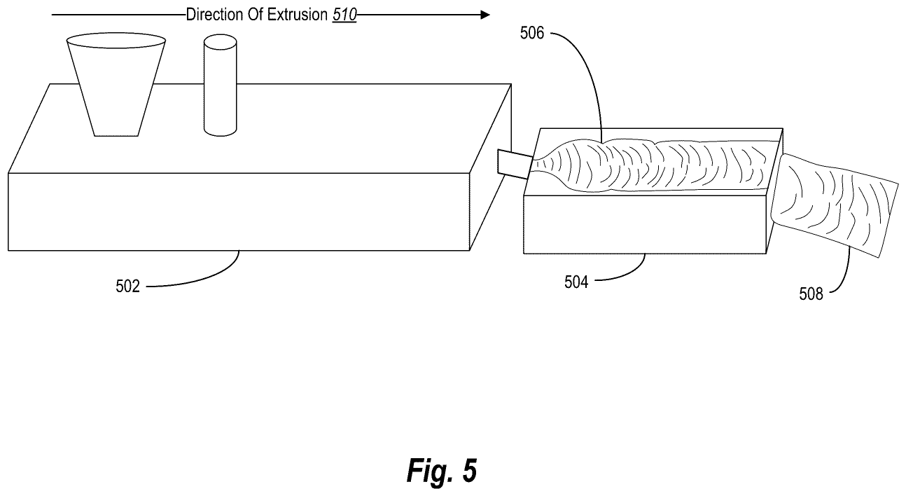

As further shown in FIG. 3, the disclosed methods can pass the cell-based dough (e.g., plant protein(s) 300, non-human cultivated animal cells 304, and marinade 308) into the cooling die 310. In some cases, the disclosed methods can rearrange and/or shape the extruded cell-based dough in the cooling die 310. As discussed in more detail with regard to FIG. 5, the disclosed methods can extrude the cell-based dough in a manner that creates a plurality of fibers that extend in a non-parallel direction relative to a direction of extrusion.

As mentioned above, the disclosed methods can regulate the temperature of a barrel of an extruder. FIG. 4 illustrates an example temperature profile of the sections of the extruder in accordance with one or more embodiments. In one example, the temperature profile depicted by FIG. 4 represents an input temperature or temperature setting. In another example, the temperature profile depicted by FIG. 4 represents a target temperature of the specific zone/portion of the extruder barrel. As discussed above, the barrel of the extruder can comprise various sections that combine and/or manipulate the proteins of one or more plant proteins and non-human cultivated animal cells. In some embodiments, the disclosed methods can modify the structure of the proteins of the one or more plant proteins and the non-human cultivated animal cells by increasing the temperature, pressure, and/or shear forces of the one or more plant proteins and the non-human cultivated animal cells.

In one or more embodiments, the disclosed methods can regulate the temperature of the barrel of the extruder by changing the temperatures of one or more sections of the barrel incrementally. For instance, in one or more embodiments, the disclosed methods can associate a temperature within a temperature range with a section of the barrel of the extruder. For example, the disclosed method can correspond a first temperature within a first temperature range to a first section 404 of the barrel of the extruder. In one or more embodiments the first temperature of the first section 404 can be between 35 and 100 degrees Celsius. In one or more implementations, the disclosed methods the first temperature of the first section 404 of the extruder can be 65 degrees Celsius.

As further shown in FIG. 4, the disclosed methods can increase the temperature of the cell-based dough. For example, the disclosed methods can pass the cell-based dough through a second section 406 of the barrel of the extruder. In some cases, the second section 406 of the extruder can correspond to a second temperature within a second temperature range to the second section 406 of the barrel of the extruder. For instance, the second temperature of the second section 406 can fall between 80 and 130 degrees Celsius. To illustrate, the disclosed methods can increase the temperature of the cell-based dough to 120 degrees Celsius once the cell-based dough passes through the second section 406.

As shown in the temperature profile, the disclosed methods can comprise a third temperature associated with a third section 408 of the barrel of the extruder. In one or more embodiments, the third temperature of the third section 408 can fall between 130 and 160 degrees Celsius. To illustrate, in one or more embodiments, the third temperature can be 155 degrees Celsius. In some implementations, the third temperature associated with the third section 408 can be a peak temperature. As used herein, the term “peak temperature” refers to the highest temperature of the barrel of the extruder. For example, the peak temperature of the barrel can be 160 degrees Celsius.

As further shown in FIG. 4 the temperature profile of the sections of the extruder can include a fourth section 410 associated with a fourth temperature within a fourth temperature range. As indicated in FIG. 4, the fourth temperature of the fourth section 410 can be lower than the third temperature (e.g., peak temperature) in the third section 408. For example, the disclosed methods can decrease the temperature of the barrel of the extruder after reaching the peak temperature. In certain cases, the fourth temperature associated with the fourth section 410 can fall between 95 and 130 degrees Celsius. To illustrate, the fourth temperature of the fourth section 410 can be 115 degrees Celsius. Relatedly, in one or more embodiments, the fourth temperature can be a melting temperature of the one or more plant proteins and non-human cultivated animal cells. As indicated in FIG. 4, the cell-based dough can pass through the fourth section 410 prior to extrusion through the cooling die 412. Thus, in some cases, the disclosed methods can increase and/or regulate the temperature of the barrel incrementally. While the temperature profile of the sections of the extruder is depicted in a step-wise fashion, in some embodiments, the disclosed methods can gradually increase and/or regulate the temperature of the extruder to fall within the temperature range associated with the section of the extruder.

In one or more embodiments, the disclosed methods can set the temperatures of the sections of the barrel based on the moisture content and/or ratios between the one or more plant proteins and the non-human cultivated animal cells, and/or water.

As further shown in FIG. 4, in one or more embodiments, the disclosed methods can add a marinade 402 to the combination of one or more plant proteins and non-human cultivated animal cells. In some embodiments, the disclosed methods can add liquid marinade through a port after the combination (e.g., cell-based dough) of one or more plant proteins and non-human cultivated animal cells drops from a peak temperature. In certain cases, the disclosed methods can mix in the marinade 402 with the combination of one or more plant proteins and non-human cultivated animal cells so that the marinade distributes substantially evenly throughout the combination of one or more plant proteins and non-human cultivated animal cells. As further shown in FIG. 4, the disclosed methods can add the marinade 402 prior to passing the marinated cell-based dough through a cooling die 412.

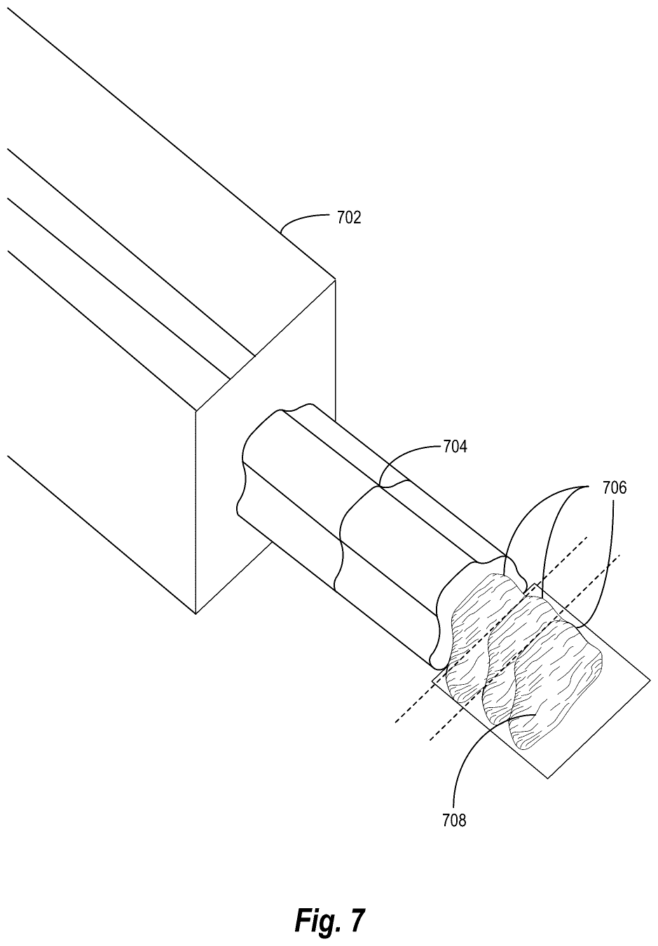

In one or more embodiments, the disclosed methods can pass the marinated non-human cultivated animal cells and one or more plant proteins through the cooling die 412. In certain embodiments, the cooling die 412 can further cool the non-human cultivated animal cells and one or more plant proteins (or marinated non-human cultivated animal cells and one or more plant proteins) as they pass one or more sections of the cooling die 412. For example, the cooling die 412 can comprise one or more cooling and/or tempering sections that can enable crosslinking between the proteins of the non-human cultivated animal cells and one or more plant proteins. As discussed below in FIG. 5 and FIG. 7, the cooling die 412 can define the shape of an extruded cell-based food product.

To reiterate, the disclosed method can extrude a combination of one or more plant proteins, non-human cultivated animal cells, glutamic acid, and/or marinade in the form of a cell-based dough into a cooling die. In one or more embodiments, the disclosed method causes the cell-based dough to travel through the cooling die in a non-parallel direction relative to a direction of extrusion. FIG. 5 illustrates the disclosed methods extruding a cell-based dough in a non-parallel direction relative to the direction of extrusion in accordance with one or more embodiments described in the present application.

As shown in FIG. 5, the disclosed methods can pass the cell-based dough from the extruder 502 through a cooling die 504 to form an extruded cell-based food product 508. In some embodiments, the disclosed methods can utilize an extrusion rate, moisture content, direction of extrusion 510 and/or viscosity of the cell-based dough and the dimensions, pressure, and/or temperature of the cooling die 504 so that a plurality of fibers 506 align orthogonally to a direction of extrusion 510 of the extruded cell-based food product 508. In one or more embodiments, the direction of extrusion 510 is the direction that the cell-based dough travels through the barrel of the extruder 502. Thus, in some embodiments, the direction of extrusion 510 is parallel to the direction and/or orientation of the barrel of the extruder 502. In one or more embodiments, the plurality of fibers 506 can align between 45 degrees and 135 degrees relative to the direction of extrusion 510.

To reiterate, the disclosed methods can regulate and/or adapt to the moisture content of the cell-based dough. In some embodiments, the disclosed methods maintain a certain moisture content of the non-human cultivated animal cells and the one or more plant proteins so that the plurality of fibers 506 align orthogonally in relation to the direction of extrusion 510. For instance, in one or more embodiments, the combination of non-human cultivated animal cells and one or more plant proteins can have a moisture content greater than 50%, or between 50% and 90%, 60% and 80%, or 70%. Relatedly, the viscosity of the combination of non-human cultivated animal cells and one or more plant proteins can be between 100,000 and 2,000,000 centipoise (cps) as they enter the cooling die 504. Additionally, the disclosed methods can utilize an extrusion rate that pushes the plurality of fibers 506 through the cooling die 504 so that the fibers have an orthogonal configuration. For instance, in one or more embodiments, the extrusion rate can be at or between 5-10 Kg/hr. In another embodiment, the extrusion rate can be at or between 10 Kg/hr-50 Kg/hr, 50-150 Kg/hr, 150-250 Kg/hr, or 250-400 Kg/hr. Higher rates are preferred at larger scales, e.g. commercial scale.

As indicated above, the disclosed methods can utilize the cooling die 504 to generate crosslinking between proteins of the combination of non-human cultivated animal cells and one or more plant proteins. As shown in FIG. 5, the disclosed method can form a plurality of fibers 506 by passing the non-human cultivated animal cells, one or more plant proteins, glutamic acid, and/or marinade through the cooling die 504. In one or more embodiments, the pressure, temperature, and/or shape of the cooling die 504 can aid in formulating the extruded cell-based food product 508. For example, in one or more embodiments, the cooling die 504 can have a narrow inlet which widens into a rectangular tunnel shape with a particular length, width, and thickness. For example, the length of the cooling die 504 can be between 50 mm and 100 mm and the width of the cooling die 504 can be between 5 mm and 40 mm. Alternatively, the width of the cooling die 504 can be between 1200 mm-1600 mm and any convenient length. In some cases, the thickness of the cooling die can range between 20 mm and 200 mm.

As shown in FIG. 5, the plurality of fibers 506 can be arranged in a direction perpendicular relative to the direction of extrusion 510. Moreover, in one or more embodiments, the plurality of fibers 506 can take on the shape of the cooling die 504. For instance, in some embodiments, the extruded cell-based food product 508 can take on a rectangular shape based on the rectangular shape of the cooling die 504.

In one or more implementations, the cooling die 504 can be pressure reducing. In some embodiments, the pressure exerted on a cell-based dough (e.g., the non-human cultivated animal cells and plant protein(s)) can fall as the cell-based dough passes through the cooling die 504 because the cell-based dough passes from a high-pressure space to a low-pressure space. Specifically, as shown in FIG. 5, the inlet to the cooling die 504 is smaller than the outlet of the cooling die. Thus, as the cell-based dough passes through the cooling die 504, the pressure exerted on the cell-based dough decreases as the cell-based dough passes from the inlet to the outlet of the cooling die 504.

As shown in FIG. 5, in certain implementations, the plurality of fibers 506 can extend in a non-parallel direction relative to a direction of extrusion 510. For example, the disclosed methods can extrude the plurality of fibers 506 into the cooling die 504 so that the plurality of fibers 506 is in an orthogonal or non-parallel direction relative to the direction of extrusion 510. Additionally, in some embodiments a first set of fibers of the plurality of fibers 506 extend in a direction orthogonal to the direction of extrusion of the extruded cell-based food product 508.

As mentioned above, in one or more embodiments, the cooling die 504 can have different cooling zones. For example, the cooling die 504 can have one or more cooling zones ranging between 30 degrees Celsius and 70 degrees Celsius, 30 degrees Celsius and 60 degrees Celsius, 35 degrees Celsius and 45 degrees Celsius, or 40 degrees Celsius for cooling and tempering the plurality of fibers 506. In certain embodiments, the one or more cooling zones have different cooling temperatures. For example, in one or more implementations, the temperature of the first cooling zone can be 60 degrees Celsius, the temperature for the second cooling zone can be 50 degrees Celsius, and the temperature for the third cooling zone can be 40 degrees Celsius. Relatedly, in one or more embodiments, the temperature of the cooling zones can be the same. In certain implementations, the disclosed methods can modify the temperature of the cooling die 504 based on the size and shape of the cooling die 504.

As noted above, the cooling die 504 can provide texture to the cell-based food product. Specifically, the cooling die 504 can cause the creation of fibers as described above. Additionally, in one or more embodiments, the interior surfaces of the cooling die 504 include a surface texture. The surface texture of the cooling die 504 can impart a corresponding texture into the outer surfaces of the cell-based food product. Additionally, the cooling die 504 can affect the internal texture of the cell-based food product. Specifically, the speed at which the cell-based dough is cooled when passing through the cooling die 504 affects the internal texture of the cell-based food product. For instance, rapid cooling can create a denser texture within the cell-based food product, while slower cooling can create a less dense cell-based food product.

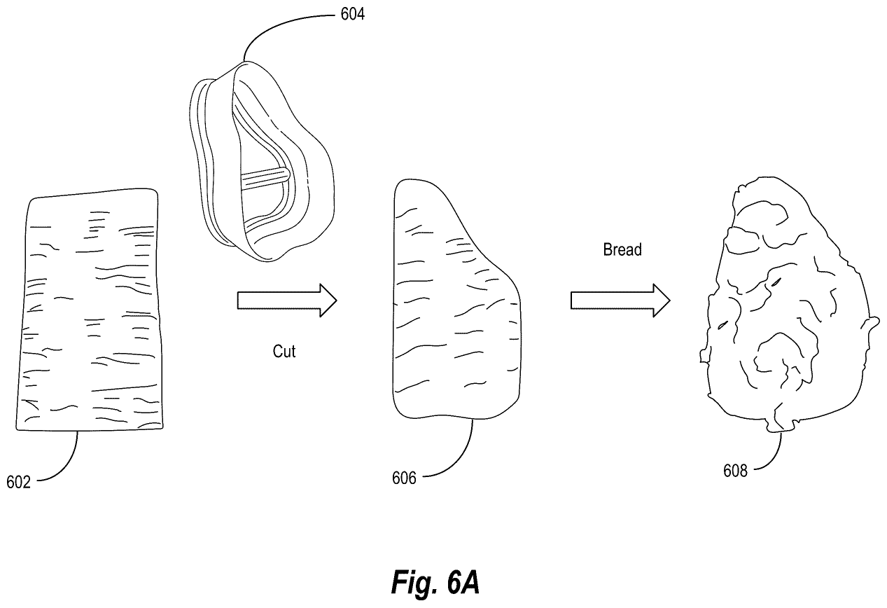

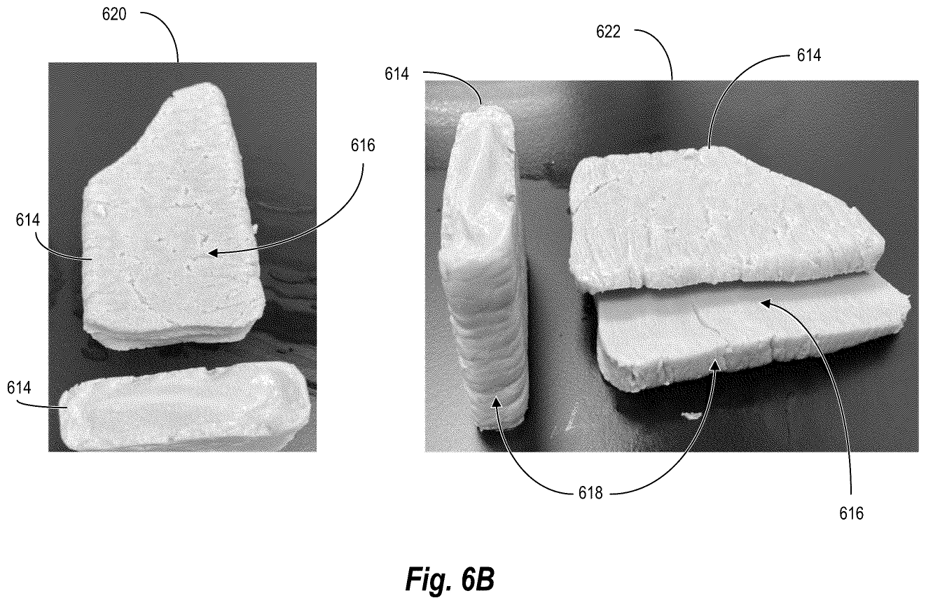

As discussed above, the extruded cell-based food product can take on the dimensions of the cooling die. FIG. 6A shows the shape and texture of an extruded cell-based food product after exiting a cooling die and further modification of the extruded cell-based food product in accordance with one or more embodiments. As shown in FIG. 6A, the extruded cell-based food product 602 has a rectangular shape based on the rectangular tunnel shape of the cooling die. Thus, in certain implementations, the extruded cell-based food product 602 can have a width, length, and thickness that corresponds to the dimensions of the cooling die. In some cases, the extruded cell-based food product 602 can have a length, width, and thickness where the length of the extruded cell-based food product 602 is longer than the width of the extruded cell-based food product 602. Additionally, in one or more embodiments, the disclosed methods can package, store, and/or cook the extruded cell-based food product 602 without modifying the shape of the extruded cell-based food product 602 after the extruded cell-based food product 602 exits the cooling die.

Alternatively, in one or more embodiments, the disclosed methods can further process and/or modify the extruded cell-based food product 602. As further shown in FIG. 6A, the disclosed method can modify the shape and/or size of the extruded cell-based food product 602. For example, as illustrated in FIG. 6A, the disclosed methods can cut the extruded cell-based food product 602 into a desired shape with a cutlet shaped die cutter 604. In particular, the disclosed methods can apply the cutlet shaped die cutter 604 to the extruded cell-based food product 602 and form an extruded cell-based food product cutlet 606 mimicking the shape of a target slaughtered meat. For example, as shown in FIG. 6A, the extruded cell-based food product 602 can have the shape of a chicken breast. In alternative embodiments, the disclosed methods can cut the extruded cell-based food product 602 into any shape that mimics the shape of a target slaughtered meat (e.g., chicken thigh, steak filet, ham cutlet, etc.). For example, the extruded cell-based food product can have a shape that mimics steak, nuggets, tenders, breasts, oysters, thighs, wings, sausage, a hamburger patty, etc. In some embodiments, when cutting the extruded cell-based food product 602 from above with a die cutter, the shape of the cut product is a mirrored shape of the die cutter. In certain embodiments, if the disclosed method cuts the extruded cell-based food product 602 from below with the die cutter, the cut product will match the shape of the die cutter.

As further shown in FIG. 6A, the disclosed method can bread the extruded cell-based food product cutlet 606 and form a breaded extruded cell-based food product 608. For example, in one or more embodiments, the disclosed methods can coat the extruded cell-based food product cutlet 606 with a dry mixture of flours, starches, seasonings, spices, salts, and/or dried herbs. In some cases, the disclosed method can further flavor and/or marinate the extruded cell-based food product cutlet 606 prior to breading. In particular, the disclosed methods can add additional marinade and/or flavorings to the extruded cell-based food product cutlet 606 prior to breading. In certain cases, the disclosed methods can further cook, heat, sear, broil, fry, and/or grill the extruded cell-based food product 602 and/or the extruded cell-based food product cutlet 606.

As discussed above, the disclosed methods can generate an extruded cell-based food product. FIG. 6B shows the texture and arrangement of the extruded cell-based food product in accordance with one or more embodiments. As shown in FIG. 6B, the first image 620 shows the external texture of the extruded cell-based food product 614. As depicted by the first image 620, the extruded cell-based food product 614 has a plurality of fibers. In particular, the first image 620 along with the second image 622 show a majority of fibers 616 of the plurality of fibers extending in a direction generally along the width of extruded cell-based food product 614. Such fibers mimic grains found in many cuts of conventional meat. Moreover, the first image 620 and second image 622 show deviations and/or changes in the direction and/or alignment of the plurality of fibers. For example, in some embodiments, a minority of fibers 618 can extend in a direction generally along the thickness of the extruded cell-based food product 614. To further illustrate, as discussed above in reference to FIG. 5, the plurality of fibers can take on an orthogonal configuration relative to a direction of extrusion. In some cases, a minority of fibers 618 of the plurality of fibers can curve or change direction while forming the extruded cell-based food product 614. The second image 622 shows the minority of fibers 618 extending in a direction generally along the thickness of the extruded cell-based food product 614 by changing direction.

As further shown in FIG. 6B the second image 622 shows an internal view of the plurality of fibers of the extruded cell-based food product 614. In particular, the second image 622 shows the plurality of fibers in a wavy or orthogonal configuration relative to the direction of extrusion where the majority of fibers 616 extend in a direction generally along the width of the extruded cell-based food product 614 and the minority of fibers 618 extend in a direction generally along the thickness of the extruded cell-based food product 614. As described above in FIG. 5, the plurality of fibers can take on an orthogonal direction relative to the direction of extrusion and mimic the texture of a slaughtered target meat. For example, FIG. 6B shows the plurality of fibers attached to each other having a muscle like appearance and texture.

In one or more embodiments, the disclosed method can modify the shape of the cooling die. FIG. 7 illustrates an alternative embodiment of the cooling die in accordance with one or more embodiments. In particular, FIG. 7 shows an extruder 702 passing the cell-based dough through a cooling die 704 having a shape mimicking a chicken cutlet. In one or more cases, the disclosed methods can modify the pressure and/or temperature of the cooling die 704 so that the fibers formed by the cell-based dough maintain an orthogonal direction relative to the direction of extrusion.

As shown in FIG. 7, in one or more embodiments, the direction of the plurality of fibers of the extruded cell-based food product 706 can change based on the shape of the cooling die 704. For example, as shown in FIG. 7, in some cases, a majority of fibers 708 can extend in a direction relative to the length of the extruded cell-based food product 706. Relatedly, as discussed above, the direction of a minority of fibers of the plurality of fibers can change as they enter the cooling die 704. For example, as described above, in one or more embodiments, the minority of fibers can extend in a direction generally along the thickness of the extruded cell-based food product 706.

As shown in FIG. 7, in one or more embodiments, the disclosed methods can slice the extruded cell-based food product 706 into to smaller sizes. For example, as the extruded cell-based food product 706 leaves the cooling die 704, the disclosed methods can slice the extruded cell-based food product 706 parallel to the plurality of fibers to a certain width.

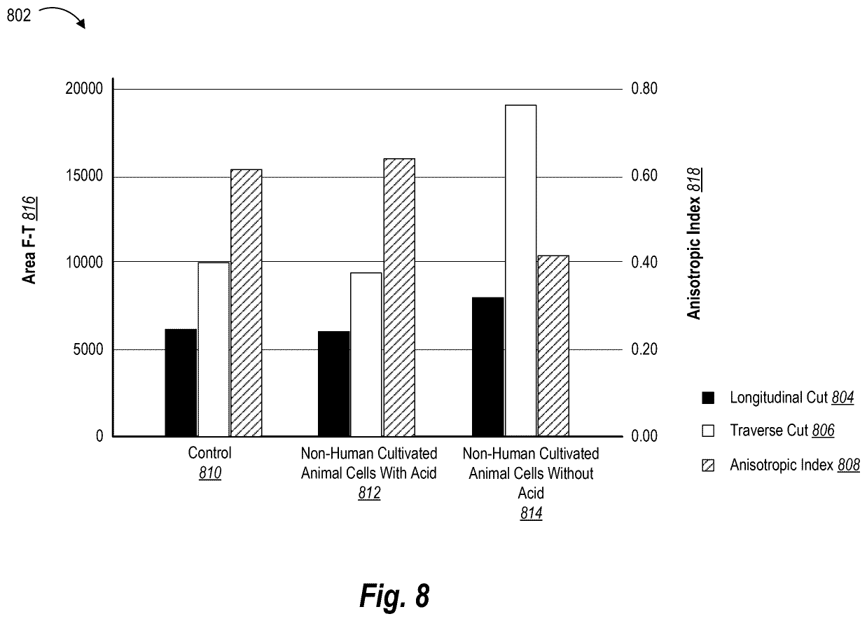

As described above, the disclosed methods can improve the texture and flavor of in-vitro cultivated food products. FIG. 8 shows an evaluation of the texture (e.g., hardness) of extruded cell-based food products, in accordance with one or more embodiments. In particular, FIG. 8 shows a comparison of the hardness of a control 810 (e.g., conventional chicken breast) with the hardness of an extruded cell-based food product comprising non-human cultivated animal cells with acid 812 (or more simply non-human cultivated animal cells with acid) and the hardness of an extruded cell-based food product comprising non-human cultivated animal cells without acid 814 (or more simply non-human cultivated animal cells without acid).

The disclosed methods utilized Warner-Bratzler methodology by measuring the area force versus time (area F-T) of a longitudinal cut 804 (e.g., cut along the grain of the product) and a traverse cut 806 (e.g., cut against the grain of the product) of the control 810 (e.g., a conventional chicken breast), the non-human cultivated animal cells with acid 812, and the non-human cultivated animal cells without acid 814. Each product has a diameter of three centimeters and a thickness of one centimeter. The disclosed method further determined the anisotropic index by calculating a ratio between the longitudinal cut 804 and traverse cut 806.

As shown in FIG. 8, graph 802 shows the area F-T 816 of the control 810, the non-human cultivated animal cells with acid 812, and the non-human cultivated animal cells without acid 814 on the left y-axis and the anisotropic index 818 on the right y-axis. As shown in FIG. 8, the area F-T 816 of the longitudinal cut 804 and the traverse cut 806 of the non-human cultivated animal cells with acid 812 is similar to the area F-T 816 of the longitudinal cut 804 and the traverse cut 806 of the control 810 (e.g., conventional chicken breast). Moreover, the graph 802 shows that the anisotropic index 808 of the non-human cultivated animal cells with acid 812 is similar to the anisotropic index 808 of the control 810 (e.g., conventional chicken breast). Additionally, the vast improvement in the longitudinal cut 804, the traverse cut 806, and the anisotropic index 808 between the non-human cultivated animal cells with acid 812 and the non-human cultivated animal cells without acid 814 shows the positive synergistic impact of acid on the texture of an extruded cell-based food product.

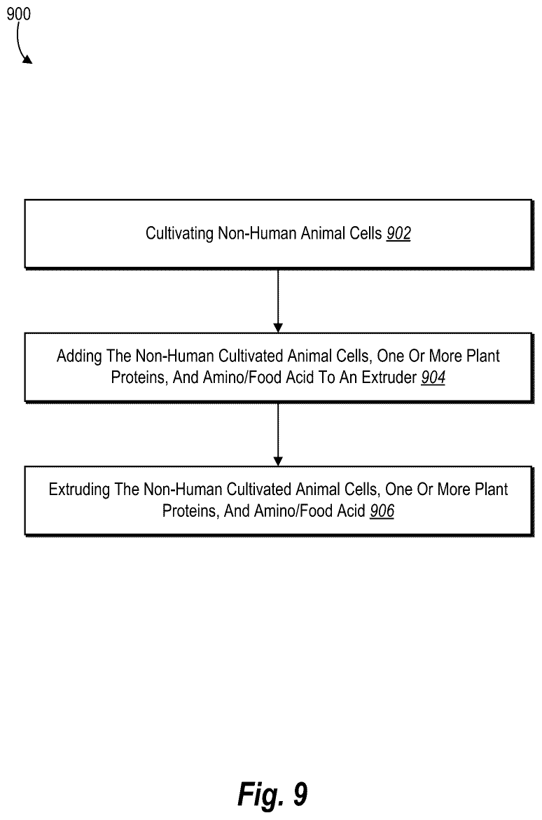

The methods, techniques, components, and/or devices used to form a cell-based food product may be used to form cell-based-foods resembling several types of slaughtered meats. FIG. 9 illustrates an example series of acts for forming an extruded cell-based food product utilizing high-moisture extrusion in accordance with one or more embodiments.

By way of overview, FIG. 9 illustrates a series of acts 900 comprising an act 902 of cultivating non-human animal cells, an act 904 of adding the non-human cultivated animal cells, one or more plant proteins, and glutamic acid to an extruder, and an act 906 of extruding the non-human cultivated animal cells, one or more plant proteins, and glutamic acid.

The series of acts 900 can include cultivating a plurality of non-human animal cells in suspension. The series of acts can further include an act of adding the plurality of non-human cultivated animal cells, one or more plant proteins, and glutamic acid to an extruder. In certain embodiments, the series of acts 900 includes an act of extruding the plurality of non-human animal cells, one or more plant proteins, and glutamic acid to create fibers therein.

In some embodiments, the series of acts 900 can include an act of cultivating a plurality of non-human animal cells in suspension. The series of acts 900 can include an act of adding the plurality of non-human cultivated animal cells and one or more plant proteins to an extruder. In one or more embodiments, the series of acts 900 can include an act of increasing a temperature and mixing the non-human animal cells and one or more plant proteins within a barrel of the extruder to form a cell-based dough. Additionally, the series of acts 900 can comprise an act of adding marinade to the cell-based dough within the barrel of the extruder after a temperature of the barrel of the extruder has dropped from a peak temperature. In one or more embodiments, the series of acts 900 can include passing the marinated cell-based dough through a cooling die.

In one or more implementations, the series of acts 900 can include an act of cultivating a plurality of non-human animal cells in suspension. In some cases, the series of acts 900 can include an act of adding the plurality of non-human cultivated animal cells and the one or more plant proteins to an extruder. In certain embodiments, the series of acts 900 can include an act of extruding the plurality of non-human animal cells and the one or more plant proteins in a manner that creates a plurality of fibers therein, wherein fibers of the plurality of fibers extend in a non-parallel direction relative to a direction of extrusion.

In certain embodiments, the series of acts 900 can include an act of adding water to the extruder. In some cases, the series of acts 900 can include an act of increasing a temperature of a barrel of the extruder. In one or more embodiments, the series of acts 900 can include an act of adding one or more of spices, flavoring agents, or salts to the extruder. In some implementations, the series of acts can include an act of adding an amount of the glutamic acid sufficient to lower a pH of the extruded cell-based food product below 6.5.

In one or more embodiments, the series of acts 900 can include an act of adding oil to the plurality of non-human cultivated animal cells and one or more plant proteins to the extruder. In one or more implementations, the series of acts 900 can include one or more of citric acid, acetic acid, lactic acid, tartaric acid, malic acid, phosphoric acid, ascorbic acid, fumaric acid, sorbic acid, or benzoic acid.

In certain implementations, the series of acts 900 can include an act of increasing the temperature of the plurality of non-human cultivated animal cells and one or more plant proteins within the barrel of the extruder further comprises increasing the temperature incrementally to the peak temperature across sections of the barrel of the extruder.

In some embodiments, the series of acts 900 can include an act where increasing the temperature incrementally to the peak temperature across sections of the barrel of the extruder comprises increasing the temperature of the barrel to a first temperature within a first section of the barrel, increasing the first temperature of the barrel to a second temperature within a second section of the barrel, and increasing the second temperature of barrel to a third temperature within a third section of the barrel. In one or more cases, the series of acts 900 can include an act where the third temperature is the peak temperature of the barrel.

In one or more embodiments, the series of acts 900 can include an act of decreasing the temperature of the barrel after reaching the peak temperature, and before passing the marinated cell-based dough through the cooling die.

In one or more embodiments, the series of acts can include an act where the direction of extrusion is parallel to the direction of a barrel of the extruder. Additionally, the series of acts 900 can include an act of mixing the plurality of non-human cultivated animal cells and the one or more plant proteins as they pass through the extruder to form a cell-based dough, and passing the cell-based dough through a cooling die. In certain cases, the series of acts 900 can include an act where a pressure exerted on the cell-based dough falls as the cell-based dough passes through the cooling die. In one or more implementations, the series of acts 900 include an act where a temperature of the cooling die ranges from 35-45 degrees Celsius.

In certain cases, the series of acts 900 can include an act of adding one or more amino acids to the extruder. Additionally, the series of acts 900 can include an act where the one or more amino acids comprise glutamic acid. In one or more implementations, the series of acts 900 can include an act where adding the one or more amino acids to the extruder comprises adding an amount of the one or more amino acids sufficient to lower a pH of the extruded cell-based food product below 6.5. In some embodiments, the series of acts 900 can include an act where the glutamic acid in combination with the plurality of non-human cultivated animal cells and the one or more plant proteins synergistically improve a texture of the extruded cell-based food product.

As described, this disclosure describes various steps to create an extruded cell-based food product and describes various embodiments of an extruded cell-based food product. In some embodiments, the extruded cell-based food product comprises non-human cultivated animal cells, one or more plant proteins, and glutamic acid, wherein the glutamic acid in combination with the non-human cultivated animal cells and the one or more plant proteins synergistically improve a texture of the extruded cell-based food product. In one or more embodiments, the extruded cell-based food product further comprises one or more of spices, flavoring, or salts.

In some embodiments, the glutamic acid comprises between 0.1% and 5% by weight of the extruded cell-based food product. In one or more cases, the non-human cultivated animal cells comprise between 1% and 65% by weight of the extruded cell-based food product. In certain embodiments, the one or more plant proteins comprise one or more of soy protein or wheat protein. In some cases, the soy protein comprises between 15% and 30% by weight of the extruded cell-based food product. Further in some instances, the wheat protein comprises between 5% and 20% by weight of the extruded cell-based food product. In some embodiments, the extruded cell-based food product has a pH between about 5.0 and 6.5.

Additionally, in one or more implementations, the glutamic acid in combination with the non-human cultivated animal cells and the one or more plant proteins synergistically improve the texture of the extruded cell-based food product by reducing a rubberiness of the extruded cell-based food product. In some embodiments, the glutamic acid in combination with the non-human cultivated animal cells and the one or more plant proteins synergistically improve the texture of the extruded cell-based food product by reducing a size of fibers in the extruded cell-based food product. In some cases, the glutamic acid comprises glutamic acid hydrochloride. In certain embodiments, the extruded cell-based food product has an anisotropic index similar to conventional chicken breast

In one or more embodiments, extruded cell-based food product comprises non-human cultivated animal cells, one or more plant proteins; and a marinade, wherein the marinade is distributed substantially throughout the extruded cell-based food product. Additionally, in some instances, the marinade comprises between 1% and 5% by weight of the extruded cell-based food product. Furthermore, in one or more cases, the marinade is not concentrated into pockets in the extruded cell-based food product. In some instances, the marinade has a greater concentration in the center of the extruded cell-based food product than the surface of the extruded cell-based food product.

Moreover, in one or more implementations, the non-human cultivated animal cells comprise between 1% and 65% by weight of the extruded cell-based food product. Also, in some cases, the one or more plant proteins comprise one or more of soy protein or wheat protein. In certain instances, the soy protein comprises between 15% and 30% by weight of the extruded cell-based food product. Additionally, in one or more embodiments, the wheat protein comprises between 5% and 20% by weight of the extruded cell-based food product.

In some implementations, the extruded cell-based food product comprises non-human cultivated animal cells, one or more plant proteins, and a plurality of fibers formed by the non-human cultivated animal cells and the one or more plant proteins, wherein one or more fibers of the plurality of fibers have a wavy configuration. In some embodiments, the extruded cell-based food product has a moisture content greater than 50%. In some cases, a first set of fibers of the plurality of fibers extend in a direction orthogonal to a direction of extrusion of the extruded cell-based food product.

In some embodiments, the extruded cell-based food product has a shape of a chicken breast, the extruded cell-based food product has a length, a width, and a thickness, and the length is longer than the width. Additionally, in one or more cases, a majority of the fibers of the plurality of fibers extend in a direction generally along the width of the extruded cell-based food product. Furthermore, in certain implementations, a minority of the fibers of the plurality of fibers extend in a direction generally along the thickness of the extruded cell-based food product. In some cases, a majority of the fibers of the plurality of fibers extend in a direction generally along the length of the extruded cell-based food product. Finally, in certain embodiments, a minority of the fibers of the plurality of fibers extend in a direction generally along the thickness of the extruded cell-based food product.

The paragraphs above describe methods for cultivating cells. FIGS. 10A-10B and the following accompanying paragraphs describe procurement of cells and growth of cells in accordance with one or more embodiments. Generally, FIGS. 10A-10B illustrate a process of collecting cells from an animal, growing cells in a favorable environment, and banking successful cells.

As illustrated by step 1002 in FIG. 10A, tissue is collected from a living animal via biopsy. In particular, stem cells, mesenchymal progeny, ectoderm lineage, and/or endoderm lineages can be isolated from the removed tissue. In some implementations of the present disclosure, tissue, such as fat and others, are processed to isolate stem cells, mesenchymal, ectoderm, and/or endoderm progeny or lineage cells. As illustrated, tissue 1004 is removed from an animal. In some examples, the tissue 1004 is removed from a living animal by taking a skin sample from the living animal. For instance, skin or muscle samples may be taken from a chicken, cow, fish, shellfish or another animal.

Cells may be extracted from the tissue 1004 that was removed from the animal. More specifically, the tissue 1004 is broken down by enzymatic and/or mechanical means. To illustrate, FIG. 10A includes digested tissue 1006 that comprises the cells to be grown in cultivation.

Cells in the digested tissue 1006 may be proliferated under appropriate conditions to begin a primary culture. As illustrated in FIG. 10A, cells 1008 from the digested tissue 1006 are spread on a surface or substrate and proliferated until they reach confluence. As shown in FIG. 10A, in some cases, cells 1012 have reached confluence when they start contacting other cells in the vessel, and/or have occupied all the available surface or substrate.

In some examples, cells are stored and frozen (i.e., banked) at different steps along the cell culture process. Cryopreservation generally comprises freezing cells for preservation and long-term storage. In some implementations, tissue and/or cells are removed from a surface or substrate, centrifuged to remove moisture content, and treated with a protective agent for cryopreservation. For example, as part of cryopreservation, tissues and cells are stored at temperatures at or below −80 C. The protective agent may comprise dimethyl sulfoxide (DMSO) or glycerol.

Cells stored through cryopreservation may be used to replenish working cell stock. For instance, while a portion of the digested tissue 1006 is used as the cells 1008 spread on a surface or substrate, the remaining or excess digested tissue 1006 is transferred to cryovials 1010 for storage. Furthermore, the cells 1012 may be banked once reaching confluence and stored in cryovials 1014.

Once the cells 1012 have reached confluence, or just before the cells 1012 have reached confluence (e.g., occupation of about 80% of the substrate), the disclosed process comprises a series of cell passage steps. During cell passage, the cells 1012 are divided into one or more new culture vessels for continued proliferation. To illustrate, the cells 1012 may be diluted or spread on one or more surfaces or substrates to form the cells 1018. The cells 1018 are then grown 1016 to confluence, or just before confluence.

The cycle of dividing the cells 1012 into the cells 1018 for continued proliferation in new culture vessels may be repeated for a determined number of cycles. Typically, cell lines derived from primary cultures have a finite life span. Passaging the cells allows cells with the highest growth capacity to predominate. In one example, cells are passaged for five cycles to meet a desired genotypic and phenotypic uniformity in the cell population.

In some implementations, the disclosed methods comprise immortalizing cells that have been grown and passaged for the determined number of cycles. For instance, the cells 1018 may be immortalized. As shown in FIG. 10B, cells 1020 have demonstrated a preferred growth capacity to proceed to immortalization. To achieve immortalization, the disclosed process transfects the cells 1020 with genes of interest. In one example telomerase reverse transcriptase (TERT) is introduced to the cells 1020. In some embodiments, the cells may be subjected to a selection process as known by those skilled in the art. The cells 1020 may then be passaged for a predetermined set of passaging cycles. In one example passaging cycle, the cells 1020 are grown to (or near) confluence 1024, then they are reseeded in new growth vessels, preserved in vials 1022, or some combination of both. The disclosed process may include any number of passaging cycles to ensure that the cells have reached immortality (e.g., can passage 60+ times without senescing), a target growth capacity, and/or a target quantity for banking. For example, cells may be passaged until they have reached a passage level of 100 (e.g., have been passaged for 100 passaging cycles). In another example, cells are passaged until they reach a population doubling level of 100.

Cells that have reached immortality or a target growth capacity by living through a target passage level may be adapted to suspension culture. In one example, a suspension culture media and agitation of cells in this suspension environment help cells to adapt and start proliferating in the new growth environment. The cells adapted to suspension 1026 may be stored in cryovials 1028 for cryopreservation and banking. Cells in suspension 1026 will begin to proliferate and the process begins a series of dilute and expand steps.

During dilution and expansion, cells are moved from growth vessels into newer, and progressively larger, growth vessels. For example, cells in suspension 1026 may begin in a single tube. The cells will proliferate and increase in cellular density. Once the cells have reached a target cell number (i.e., viable cell density (VCD) at desired volume), they are diluted and moved to a larger growth vessel. Optionally, the cells are banked in cryovials throughout expansion. For example, once cells in suspension reach a maximum VCD, the cells may begin to leave exponential growth due to overcrowding. After reaching a target density, the suspension cells may be transferred to a larger vessel 1030 and diluted with additional media. The dilute-and-expand steps are repeated using progressively larger vessels (e.g., the vessel 1031 and the vessel 1032) and/or progressive dilution until the cells reach a production-ready volume. For example, cells may be production ready at about a 1,000-100,000 liter scale at 5 million cells per mL. The cells may be banked in cryovials at any of the dilution and expansion cycles. In one or more embodiments, the cells may be further processed in an extruder or other mechanical apparatus that manipulates or changes the structure and/or arrangement of protein in the cells. In some embodiments, the cells may be processed before extrusion. For example, the disclosed method can dilute the cells to a specific cell density or consistency prior to extrusion.

In accordance with common practice, the various features illustrated in the drawings may not be drawn to scale. The illustrations presented in the present disclosure are not meant to be actual views of any particular apparatus (e.g., device, system, etc.) or method, but are merely idealized representations that are employed to describe various embodiments of the disclosure. Accordingly, the dimensions of the various features may be arbitrarily expanded or reduced for clarity. In addition, some of the drawings may be simplified for clarity. Thus, the drawings may not depict all of the components of a given apparatus (e.g., device) or all operations of a particular method.

Terms used herein and especially in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” etc.).

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” or “one or more of A, B, and C, etc.” is used, in general such a construction is intended to include A alone, B alone, C alone, A and B together, A and C together, B and C together, or A, B, and C together, etc. For example, the use of the term “and/or” is intended to be construed in this manner.

Further, any disjunctive word or phrase presenting two or more alternative terms, whether in the description, claims, or drawings, should be understood to contemplate the possibilities of including one of the terms, either of the terms, or both terms. For example, the phrase “A or B” should be understood to include the possibilities of “A” or “B” or “A and B.”

However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

Additionally, the use of the terms “first,” “second,” “third,” etc., are not necessarily used herein to connote a specific order or number of elements. Generally, the terms “first,” “second,” “third,” etc., are used to distinguish between different elements as generic identifiers. Absent a showing that the terms “first,” “second,” “third,” etc., connote a specific order, these terms should not be understood to connote a specific order. Furthermore, absent a showing that the terms “first,” “second,” “third,” etc., connote a specific number of elements, these terms should not be understood to connote a specific number of elements. For example, a first widget may be described as having a first side and a second widget may be described as having a second side. The use of the term “second side” with respect to the second widget may be to distinguish such side of the second widget from the “first side” of the first widget and not to connote that the second widget has two sides.

As used herein, the term “substantially,” in reference to a given parameter, property, or condition, means to a degree that one of ordinary skill in the art would understand that the given parameter, property, or condition is met within a degree of variance, such as within acceptable manufacturing tolerances. By way of example, depending on the particular parameter, property, or condition that is substantially met, the parameter, property, or condition may be at least 70.0% met, at least 80.0%, at least 90% met, at least 95.0% met, at least 99.0% met, or even at least 99.9% met.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present disclosure have been described in detail, it should be understood that the various changes, substitutions, and alterations could be made hereto without departing from the spirit and scope of the present disclosure.

The present invention may be embodied in other specific forms without departing from its spirit or essential characteristics. Indeed, the described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel to one another or in parallel to different instances of the same or similar steps/acts. The scope of the invention is, therefore, indicated by the appended claims rather than by the foregoing description. All changes that come within the meaning and range of equivalency of the claims are to be embraced within their scope.

What is claimed is:

1. A method of forming an extruded cell-based food product comprising:
cultivating a plurality of non-human animal cells in suspension;
adding the plurality of non-human cultivated animal cells, one or more plant proteins, and glutamic acid to an extruder; and
extruding the plurality of non-human cultivated animal cells, one or more plant proteins, and glutamic acid to create fibers therein.

2. The method of claim 1, further comprising adding water to the extruder.

3. The method of claim 1, further comprising increasing a temperature of a barrel of the extruder.

4. The method of claim 1, further comprising adding one or more of spices, flavoring agents, or salts to the extruder.

5. The method of claim 1, further comprising adding an amount of the glutamic acid sufficient to lower a pH of the extruded cell-based food product below 6.5.

6. The method of claim 1, further comprising adding oil to the plurality of non-human cultivated animal cells and one or more plant proteins to the extruder.

7. The method of claim 1, further comprising adding one or more of citric acid, acetic acid, lactic acid, tartaric acid, malic acid, phosphoric acid, ascorbic acid, fumaric acid, sorbic acid, or benzoic acid.

8. A method of forming an extruded cell-based food product comprising:
cultivating a plurality of non-human animal cells in suspension;
adding the plurality of non-human cultivated animal cells and one or more plant proteins to an extruder;
increasing a temperature and mixing the plurality of non-human cultivated animal cells and one or more plant proteins within a barrel of the extruder to form a cell-based dough;
adding marinade to the cell-based dough within the barrel of the extruder after a temperature of the barrel of the extruder has dropped from a peak temperature; and
passing the marinated cell-based dough through a cooling die.

9. The method of claim 8, wherein increasing the temperature of the plurality of non-human cultivated animal cells and one or more plant proteins within the barrel of the extruder further comprises:
increasing the temperature incrementally to the peak temperature across sections of the barrel of the extruder.

10. The method of claim 9, wherein increasing the temperature incrementally to the peak temperature across sections of the barrel of the extruder comprises:
increasing the temperature of the barrel to a first temperature within a first section of the barrel;
increasing the first temperature of the barrel to a second temperature within a second section of the barrel; and
increasing the second temperature of barrel to a third temperature within a third section of the barrel.

11. The method of claim 10, wherein the third temperature is the peak temperature of the barrel.

12. The method of claim 8, further comprising:
decreasing the temperature of the barrel after reaching the peak temperature; and
before passing the marinated cell-based dough through the cooling die.

13. A method of forming an extruded cell-based food product comprising:
cultivating a plurality of non-human animal cells in suspension;
adding the plurality of non-human cultivated animal cells and one or more plant proteins to an extruder; and
extruding the plurality of non-human cultivated animal cells and the one or more plant proteins in a manner that creates a plurality of fibers therein, wherein fibers of the plurality of fibers extend in a non-parallel direction relative to a direction of extrusion.

14. The method of claim 13, further comprising:
mixing the plurality of non-human cultivated animal cells and the one or more plant proteins as they pass through the extruder to form a cell-based dough; and
passing the cell-based dough through a cooling die.

15. The method of claim 14, wherein a pressure exerted on the cell-based dough falls as the cell-based dough passes through the cooling die.

16. The method of claim 14, wherein a temperature of the cooling die ranges from 35-45 degrees Celsius.

17. The method of claim 13, further comprising adding one or more amino acids to the extruder.

18. The method of claim 17, wherein the one or more amino acids comprise glutamic acid.

19. The method of claim 18, wherein adding glutamic acid in combination with the plurality of non-human cultivated animal cells and the one or more plant proteins synergistically improve a texture of the extruded cell-based food product.

20. The method of claim 17, wherein adding the one or more amino acids to the extruder comprises adding an amount of the one or more amino acids sufficient to lower a PH of the extruded cell-based food product below 6.5.
